(19) United States
(12) Patent Application Publication
(10) Pub. No.: US 2004/0228928 A1
(43) Pub. Date: Nov. 18, 2004

(54) Method of altering, enhancing and standardizing the potency and bioactivity of bioactive polysaccharides

(76) Inventor: Arthur William Zeigler, Raymond, WA (US)

Correspondence Address: ARTHUR WILLIAM ZEIGLER, 231 11th STREET, PO BOX 6, RAYMOND, WA, 98577-0006, US

(21) Appl. No.: 10/769,328

(22) Filed: Jan. 30, 2004

Related U.S. Application Data
(60) Provisional application No. 60/471,287, filed on May 15, 2003.

(51) Int. Cl. A61K 33/24; A61K 31/715

(52) U.S. Cl. 424/617; 514/54

(57) ABSTRACT

A METHOD OF ALTERING, POTENTIZING, STANDARDIZING AND ENHANCING THE BIOACTIVITY OF BIOACTIVE POLYSACCHARIDES with selected minerals, either singly or in combination, used together with a bioactive polysaccharide for additive and/or synergistic alteration of the bioactivity of the polysaccharide.

### CROSS REFERENCE TO RELATED APPLICATIONS

This application is based on provisional application serial number 60/471,287, filed on May 15, 2003.

### STATEMENT REGARDING FEDERALLY SPONSORED RESEARCH OR DEVELOPMENT

Not Applicable

### DESCRIPTION OF ATTACHED APPENDIX

Not Applicable

### BACKGROUND OF THE INVENTION

This invention relates generally to the field of bioactive polysaccharides and more specifically to a METHOD OF ALTERING, ENHANCING AND STANDARDIZING THE POTENCY AND BIOACTIVITY OF BIOACTIVE POLYSACCHARIDES AND THE CREATION OF VARIED AND/OR NEW BIOACTIVE PROPERTIES.

Decades of research and thousands of years of folk medicine usage have established that numerous plants contain bioactive polysaccharides such as those found in Aloe Vera, Echinacea, certain mushrooms and Larch trees, to name but a few of many. Examples of these polysaccharides known to have healing properties are: Mucopolysaccharides from Aloe Vera, Beta-Glucan from certain mushrooms and baker's yeast, also arabinogalactin from Echinacea and Larch trees.

Bioactive polysaccharide containing herbs and herbal extracts are notoriously difficult to standardize as to potency and effective dosage. This has led to the industry wide practice of bioactive polysaccharide extraction and refination to states of higher concentration and purity.

These polysaccharides, in their natural state, are associated mechanically, electrostaticly, by valance bonding or otherwise with minerals. Review of published assays for Aloe Vera extract in various states of purification indicated that, as the extract became more refined, mineral elements diminished as a constituent. Confirmatory assays performed by the inventor verified the above observation.

The inventor postulated that a portion of the historically proven health benefits of bioactive plants containing polysaccharides are, in fact the action of those polysaccharides in association with minerals.

The inventor claims that the potency of bioactive polysaccharides can be altered, potentized, standardized and enhanced by the addition of select minerals, either singly or in combination.

U.S. Pat. No. 5,294,434

is an example of a bioactive polysaccharide used together with minerals, each for the separate purposes of their individual properties rather than an attempt at achieving synergy.

The Aloe Vera polysaccharides are used as bactericidal healing agents and the minerals are used as an abrasive dentifrice.

This is an example of an effective combination of polysaccharide and mineral but it does not take advantage of the synergy which could be achieved by combining the Aloe Vera polysaccharides with selected minerals for the purpose of increasing its their effectiveness.

U.S. Pat. No. 5,770,217

In this patent a number of bioactive polysaccharide containing herbs are used together with minerals but not for the purpose of altering the bioactivity of the polysaccharides. Selenium, for example, is used to potentize the antioxidant effects of vitamins and other minerals are used for their nutritive value. Had these inventors understood the potential benefits achievable by the appropriate selection of minerals for enhancing the effects of the bioactive polysaccharides, their compounds could have been much more effective.

U.S. Pat. No. 5,880,109

is an example of a bioactive polysaccharide used to accelerate the intestinal absorption of calcium. While this is a good use, it does not recognize that a mineral can be used to alter the bioactivity of the bio active polysaccharides.

U.S. Pat. No. 6,632,459

pertains to compounds of various bioactive polysaccharides together with various minerals and chlorogenic acid or an analog thereof. In all cases, the bioactive polysaccharides and the minerals are used for their individual properties, rather than an enhancement of biactivity on the part of the bioactive polysaccharides. Had this inventor realized the potential synergies to be achieved, these formulations could have been enhanced by the appropriate selection of minerals to increase the potency of the various contained bioactive polysaccharides.

U.S. Pat. No. 6,551,596

Is an example of bioactive polysaccharides used together with minerals and vitamins. The only stated purpose of the mineral additives is to provide a complete formula diet to be used as a sole source of nutrition, which can supply trace element nutritional requirements. With the understanding of the current invention these formulations could have been made more bioactive. In the past, minerals have been added to compounds containing bioactive polysaccharides to perform various independent functions of their own in parallel with the actions of the bioactive polysaccharides. These usages have not recognized the enormous potential for altering, potentizing, enhancing and standardizing the bioactive properties of the bioactive polysaccharides by utilizing the additive and/or synergistic effects of the mineral additives. The current invention does recognize and utilize those benefits.

### BRIEF SUMMARY OF THE INVENTION

The primary object of the invention is to enhance the potency of bioactive polysaccharides by the addition of selected minerals, either singly or in combination. This is achieved by additive and/or synergistic effects.

Another object of the invention is to restore the potency of over-refined bioactive polysaccharides by the addition of selected minerals, either singly or in combination.

Another object of the invention is adding measured amounts of selected minerals, either singly or in combination, to standardize the effects of bioactive polysaccharides.

A further object of the invention is to give varied and/or new properties to bioactive polysaccharides by the addition of selected minerals either singly or in combination. This is achieved by additive and/or synergistic effects.

Yet another object of the invention is that the bioactive polysaccharides contained in herbs and herbal preparations can have their bioactivity altered, enhanced and/or standardized in like manner by the addition of selected minerals either singly or in combination.

Other objects and advantages of the present invention will become apparent from the following descriptions, taken in connection with the accompanying Tables 1 & 2, wherein, by way of illustration and example, an embodiment of the present invention is disclosed.

In accordance with a preferred embodiment of the invention, there is disclosed a METHOD OF ALTERING AND ENHANCING THE POTENCY AND BIOACTIVITY OF BIOACTIVE POLYSACCHARIDES comprising: Selected minerals, either singly or in combination, are used together with a bio-active polysaccharide for additive and/or synergistic alteration of the bioactivity of the polysaccharide, different minerals, forms of minerals and/or blends of minerals used together with a bioactive polysaccharide can cause the bioactivity of the polysaccharide to be enhanced, and

In accordance with a preferred embodiment of the invention, there is disclosed a METHOD OF ALTERING AND STANDARDIZING THE POTENCY AND BIOACTIVITY OF BIOACTIVE POLYSACCHARIDES comprising: Selected minerals, either singly or in combination, are used together with a bioactive polysaccharide for additive and/or synergistic alteration of the bioactivity of the polysaccharide, measured amounts of different minerals, forms of minerals and/or blends of minerals used together with a quantity of a bio-active polysaccharide can cause the bioactive polysaccharide to produce consistent and standardized effects, and

In accordance with a preferred embodiment of the invention, there is disclosed a METHOD OF ALTERING AND CREATING VARIED AND/OR NEW BI-ACTIVE PROPERTIES FOR BIOACTIVE POLYSACCHARIDES comprising: Selected minerals, either singly or in combination, are used together with a bioactive polysaccharide for additive and/or synergistic alteration of the bioactivity of the polysaccharide, different minerals, forms of minerals and/or blends of minerals used together with a bioactive polysaccharide can cause the bioactive polysaccharide to produce varied and/or new effects.

### DETAILED DESCRIPTION OF THE PREFERRED EMBODIMENTS

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

As noted above, this invention pertains to the use of minerals together with a bioactive polysaccharide in order to alter, enhance, potentize and standardize the bioactivity of the polysaccharide. This use of minerals with a polysaccharide may also endow it with new and/or varied attributes. An analogy can be drawn to the use of trace minerals in the electronics industry for the doping of silicon wafers to endow them with new properties.

As used in this disclosure, the term “mineral” refers to inorganic substances, metals, metal salts and others, which are nutritionally beneficial and others which are known to those of ordinary skill in the art. These minerals may be in different forms such as, but not limited to, mineral elements, mineral salts, atomic minerals, diatomic minerals, mineral micro-clusters, mineral colloids, ionic minerals, chelated minerals and other forms of minerals used singly or in combination.

Such preparations of mineral enhanced polysaccharide may be in many forms such as but without limitation, pills, liquids, lotions, ointments and bioactive cosmetics and personal care products.

The preparation may comprise polysaccharide containing plant material in raw form and may also be administered as an herbal tea. This may be advantageous, in particular to lower the cost of a preparation.

Bioactive polysaccharides come from many sources and are available in a wide range of purities and states of refination. They occur in plants, herbs, seaweed, fungi, yeast and man made and cultured products.

A person with ordinary skill in the art can select a polysaccharide with a desired proven or suspected bio-activity and this may be altered, enhanced, potentized and/or standardized by the addition of selected minerals either singly or in combination

Examples of suitable polysaccharides are mucopolysaccharides from Aloe Vera and arabinogalactans from Baptisa tinctoria, Echinacea species, Larix occidentalis, Angelica acutiloba, Thuja occidentalis and Panix ginsing. Other useful bio-active polysaccharides are contained in Arnica montana cell cultures, Plantago spp, Achyrocline satureioides, Aconitum officinalis, Angelica acutiloba, Aristolochia officinalis, Astralagus gummerifa, A. membranaceus, A. mongholicus, Avena sativa, Bambusa vulgaris, Baptisia tinctoria, Bryonia dioica, Calendula officinalis, Carthamus tinctorius, Chamomilla recutita, Echinacea angustifolia, E. pallida, E. purpurea, Eleutherococcus senticosus, Eupatorium cannabinum, Morinda citrifolia, Silene vulgaris, Thuja occidentalis, Triticum sativum, Vincetoxicum officinalis and Viscum album.

Polysaccharides from fungi, in particular from Flammulina velupites, Lentinus edodes, Schizophyllum commune, Phytophtora parasytica, Grifola frondosa, Sclerotinia clerotiorum, Ganoderma lucidum, Coriolus versicolor and the like may also be used.

Naturally a preparation according to the invention may also comprise one or more bioactive polysaccharides not listed above.

A preferred preparation according to the present invention comprises one or more bioactive polysaccharides and one or more minerals selected to alter the bioactivity of the polysaccharide.

A preferred embodiment of the preparation comprises an extract of one or more plants, such as plant extracts from whole leaf Aloe Vera and Larix occidentalis and the like. These extracts may be alcoholic and/or aqueous extracts or prepared according to any method but preferably standardized as to polysaccharide content.

The extracts may be dried, or mixed in a liquid or prepared in another manner and may provide various compounds including polysaccharides which have been found to be bioactive.

Particularly good results have been achieved with preparations comprising mucopolysaccharides extracted from whole leaf Aloe Vera and arabinogalactans extracted from Larix occidentalis combined together with mineral extracts from seawater, dolomite, lava, and fulvic shale. Other minerals and sources of minerals could be used.

A preparation, according to the invention, can be widely used for a variety of reasons and by a variety of subjects of any age and of various species including humans. The concentration of a substance present in a preparation, according to the invention, may vary widely based upon several factors. These factors include the species to be treated (e.g. cattle, a pet or a human), the condition of the subject to be treated, the reason for administration (e.g. a particular diagnosis or prophylaxis), or the form of preparation (e.g. a concentrated tablet, dissolved in a drink or mixed in cattle or pet feed). In a preferred embodiment, a preparation comprises the substances shown in Table 1 in an aqueous preparation.

TABLE 1
The mineral enhanced polysaccharide formula contains
10,000 parts per million of freeze dried Aloe Vera
polysaccharides and 40,000 parts per million of purified
larch tree arabinogalactin in deionized water to which was
added minerals extracted from sea water, dolomite, lava
and fulvic shale. The minerals are in the form of hydroxides,
oxides and salts. The following is a listing of those minerals
which are detectable in the formula at the level of .1 Parts
Per Million or higher by Inductively Coupled Mass Spectroscopy.

| | | |
|---|---|---|
| Aluminum | 11.77 | PPM |
| Barium | 1.25 | PPM |
| Boron | 6.01 | PPM |
| Bromine | 0.32 | PPM |
| Calcium | 1,331.18 | PPM |
| Cesium | 3.0 | PPM |
| Chloride | 343.0 | PPM |
| Copper | 1.47 | PPM |
| Iron | 62.47 | PPM |
| Magnesium | 3,208.57 | PPM |
| Manganese | 4.0 | PPM |
| Neodymium | 0.2 | PPM |
| Nickel | 0.2 | PPM |
| Phosphorus | 84.98 | PPM |
| Potassium | 361.19 | PPM |
| Silicon | 61.82 | PPM |
| Sodium | 1,305.62 | PPM |
| Strontium | 8.95 | PPM |
| Sulfur | 19.05 | PPM |
| Tin | 1.75 | PPM |
| Titanium | 0.22 | PPM |
| Vanadium | 0.20 | PPM |
| Yttrium | 0.22 | PPM |
| Zinc | 3.3 | PPM |

The daily dose of this preparation is 5 to 15 milliliters per day. Herein “daily dose” is used to subscribe a daily dose for a primate having a body weight 70 kg, unless stated otherwise. Methods to calculate the daily dose for other subjects will be known to the skilled professional. Once the daily dose is determined it can be calculated how much of a certain preparation needs to be administered, knowing the (approximate) fraction of active components and additives in the preparation.

In animal testing conducted by the inventor, the above formula was used to supplement the diet of white mice for a period of 30 days after which they were subjected to forced swim testing. The test population, which received the supplementation of mixed, bioactive polysaccharides enhanced with minerals, exhibited more stamina than the unsupplemented control group by enduring the forced swim testing for a duration 2.52 times greater. The test group receiving the mineral enhanced polysaccharide blend exhibited more stamina than the group receiving polysaccharides only by enduring the forced swim testing for a duration 1.3 times greater.

This illustrates and proves the claims of this invention since the minerals added to the bioactive polysaccharides in the preferred embodiment listed above can not by themselves account for the enhanced stamina of the test population which received the preparation as a supplement. The greatly increased vigor of this group, compared to the control group, which received no supplementation and the test population, which received the polysaccharides only, establishes that there is an additive and/or synergistic effect when the appropriate minerals are added to a bioactive polysaccharide. This effect can be used to alter, enhance, standardize and give new properties to the bioactive polysaccharides.

An accident, which occurred during another animal test, gives a further illustration of the efficacy of this preparation. Two populations of white mice were placed in a hyperbaric chamber at the same time and were supplied with Oxygen while under ½ atmosphere of pressure. One population had received, as a supplement to their diet for seven days, the complete preparation as listed above. The other group had received supplementation with the mineral portion of the preparation only and for an equal length of time.

Inadvertently the Oxygen was over supplied which caused, over the next 48 hours, an 80% mortality in the population which had received supplementation with minerals only. The other group, which had received supplementation with the complete preparation was unscathed. In fact, three weeks later when the experiment was terminated this group had one of the highest scores for stamina during forced swim testing when compared against six other populations.

It is a further advantage of the invention that the bioactive polysaccharides and other components comprised by the preparation are often natural products, which have substantially no negative side effects upon administration. This makes it possible to market the preparation as an over the counter drug, which means that it can be sold without prescription of a physician. Moreover it has been found that it is possible to process preparations according to the invention in food & drinks products, which may further facilitate the accessibility of products of the invention to the public, which more and more becomes aware of the importance of health-food products as an aid to maintain one's general health.

For the manufacture of a product for promoting vigor and stamina, a preparation according to the invention may be used. The preparation may be processed by methods known from the pharmaceutical and food & drinks processing industries into a pharmaceutical, a drink or a food product.

In a preferred embodiment the preparation may be in a more or less solid form such as a capsule, a tablet, a lozenge, a powder, an agglomerate, a snack, a bar or a paste. The preparation may also be a solution, a liquid or a semi-liquid shaped product, such as a gel, an emulsion, a suspension, a drink (such as a lemonade, a fruit juice, a dairy drink, a pudding, an ice cream, a sauce and the like.

In a preferred embodiment the preparation may be rendered suitable for external application as wound dressings, poultices, salves, bio-active cosmetics and personal care products and the like.

A preparation may be processed in a drink or food product at the factory. A drink, comprising a daily dose may for example be packed in a 250 ml package, or another volume that is suitably consumed by a subject. In another embodiment the active components are present in a bar of typically up to 25 g, which may further comprise typically at least 60% of (sweet) carbohydrates. In another embodiment, a preparation, e.g. a powder, a liquid, a suspension and the like, may be administered to a drink or food, such as a dessert or a juice, relatively shortly before oral intake. Such a preparation may for example substantially consist of active components and may in principle be administered by anyone.

The preparation may also be a complete nutritive food product, e.g. for patients on a particular (strict) diet or for cancer patients.

In a preferred method for stimulating the immune system, a preparation according to the invention is administered to a patient, in the form of a capsule, a tablet, a powder or a lozenge. In another embodiment the preparation may be administered in another form such as drops or a potion.

For the formulation of the dosage form, the above described preparation may suitably be combined with a pharmaceutically acceptable carrier. A much preferred dosage form is the tablet, in which the preparation may be combined with usual tablet constituents such as filler-binders, lubricants and the like.

The dosage in which the preparation is to be administered will of course depend on the concentration of active ingredients of the preparation and on the severity of the medical disorder or disease that is to be treated or prevented. The person skilled in the art will be able to optimize and formulate a suitable dosage scheme based on his ordinary knowledge of the field.

It is an advantage of the invention that standardization of potency, by using measured amounts of a mineral or minerals with a measured quantity of a bioactive polysaccharide or polysaccharides according to the preferred embodiment of the invention, allows for a more precise and efficacious application of dosage.

Preferably the preparation is orally administered, although other administration methods known in the art may also be applied, for some embodiments.

The packaging preferably occurs to facilitate convenience of packaging, storage, transport and/or consumption, and to ensure stability of the product, using methods known in the art.

The invention will now be further elucidated by the following, non-restrictive example.

To overcome chemical instability and oxidation of the polysaccharide content of a preparation, a preferred embodiment of a preparation prepared according to the invention would utilize a preservative system which would be compatible with both the polysaccharide content and the mineral content of the preparation. In a preferred embodiment, a preparation comprises the substances shown in Table 2 in an aqueous preparation.

TABLE 2
The mineral enhanced polysaccharide formula contains
10,000 parts per million of freeze dried Aloe Vera
polysaccharides and 40,000 parts per million of purified
larch tree arabinogalactin in deionized water to which was
added minerals extracted from sea water, dolomite, lava
and fulvic shale. The minerals are in the form of hydroxides
and salts. The following is a listing of those minerals that are
detectable in the formula at the level of .1 Parts Per Million or
higher by Inductively Coupled Mass Spectroscopy.

| | | |
|---|---|---|
| Aluminum | 11.77 | PPM |
| Barium | 1.25 | PPM |
| Boron | 6.01 | PPM |
| Bromine | 0.32 | PPM |
| Calcium | 1,331.18 | PPM |
| Cesium | 3.0 | PPM |
| Chloride | 343.0 | PPM |
| Copper | 1.47 | PPM |
| Iron | 62.47 | PPM |
| Magnesium | 3,208.57 | PPM |
| Manganese | 4.0 | PPM |
| Neodymium | 0.2 | PPM |
| Nickel | 0.2 | PPM |
| Phosphorus | 84.98 | PPM |
| Potassium | 361.19 | PPM |
| Silicon | 61.82 | PPM |
| Sodium | 1,305.62 | PPM |
| Strontium | 8.95 | PPM |
| Sulfur | 19.05 | PPM |
| Tin | 1.75 | PPM |
| Titanium | 0.22 | PPM |
| Vanadium | 0.20 | PPM |
| Yttrium | 0.22 | PPM |
| Zinc | 3.3 | PPM |

The pH of the mineral blend is lowered to between 5.5 and 6.0 with citric acid and the polysaccharides are added to the blend. Potassium sorbate is then added at the rate of 3/10 of one percent. Finally, ascorbic acid is added at the rate of ½ of one percent of the total blend by weight.

With respect to all methods disclosed herein, all steps, procedures or processes may be considered to be done without regard to the priority unless specified, or dictated by necessity or force of logic. The identification of steps or products in any priority is done only for the purposes of introducing them and distinguishing them from one another, unless otherwise dictated by necessity or force of logic.

The foregoing is offered primarily for purposes of illustration. It will be readily apparent to those skilled in the art that the proportions, materials, formulation procedures, administration protocols and other parameters of this invention may be further modified or substituted in various ways without departing from the spirit and scope of the invention.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims.

What is claimed is:

1. A METHOD OF ALTERING AND ENHANCING THE POTENCY AND BIOACTIVITY OF BIOACTIVE POLYSACCHARIDES comprising:
Selected minerals, either singly or in combination, are used together with a bioactive polysaccharide for additive and/or synergistic alteration of the bioactivity of the polysaccharide;
whereby a mineral, different minerals, different forms of minerals and/or blends of minerals used together with a bioactive polysaccharide can cause the bioactivity of the polysaccharide to be substantially enhanced; and

2. A MEANS UNDER CLAIM 1 OF ALTERING AND ENHANCING THE POTENCY AND BIOACTIVITY OF PURIFIED BI-ACTIVE POLYSACCHARIDES comprising:
Selected minerals, either singly or in combination, are used together with a purified bioactive polysaccharide for additive and/or synergistic alteration of the bio-activity of the purified polysaccharide;
whereby a mineral, different minerals, different forms of minerals and/or blends of minerals used together with a purified bioactive polysaccharide can cause the bioactivity of the purified polysaccharide to be substantially enhanced; and

3. A MEANS UNDER CLAIM 1 OF ALTERING AND ENHANCING THE POTENCY AND BIOACTIVITY OF MAN MADE OR CULTURED BIOACTIVE POLYSACCHARIDES comprising:
Selected minerals, either singly or in combination, are used together with a man made or cultured bioactive polysaccharide for additive and/or synergistic alteration of the bioactivity of the man made or cultured polysaccharide;
whereby a mineral, different minerals, different forms of minerals and/or blends of minerals used together with a man made or cultured bioactive polysaccharide can cause the bioactivity of the man made or cultured polysaccharide to be substantially enhanced; and

4. A MEANS UNDER CLAIM 1 OF ALTERING AND ENHANCING THE POTENCY AND BIOACTIVITY OF BIOACTIVE POLYSACCHARIDES CONTAINED IN HERBS AND PLANTS comprising:
Selected minerals, either singly or in combination, are used together with an herb or plant containing a bioactive polysaccharide for additive and/or synergistic alteration of the bi-activity of the polysaccharide contained in said herb or plant;
whereby a mineral, different minerals, different forms of minerals and/or blends of minerals used together with an herb or plant containing a bioactive polysaccharide can cause the bioactivity of the polysaccharide to be substantially enhanced; and

5. A MEANS UNDER CLAIM 1 OF ALTERING AND ENHANCING THE POTENCY AND BIOACTIVITY OF BIOACTIVE POLYSACCHARIDES comprising:
Selected minerals, either singly or in combination, are used together with a bioactive polysaccharide for additive and/or synergistic alteration of the bioactivity of the polysaccharide;
whereby a mineral, different minerals or different forms of minerals such as, but not limited to, mineral elements, mineral salts, mineral colloids, mineral micro-clusters, atomic minerals, diatomic minerals, Ionic minerals, chelated minerals and other forms of minerals used singly or in combination, together with a bioactive polysaccharide can cause the bioactivity of the polysaccharide to be substantially altered and/or substantially enhanced; and

6. A MEANS UNDER CLAIM 1 OF ALTERING AND ENHANCING THE POTENCY AND BIOACTIVITY OF BIOACTIVE POLYSACCHARIDES FOR HUMAN AND ANIMAL USE comprising:
Selected minerals, either singly or in combination, are used together with a bioactive polysaccharide for additive and/or synergistic alteration of the bioactivity of the polysaccharide;
whereby a mineral, different minerals, different forms of minerals and/or blends of minerals used together with an herb or plant containing a bioactive polysaccharide can cause the bioactivity of the polysaccharide to be substantially enhanced; and such an enhanced bioactive polysaccharide would then be suitable for nutritive and/or medicinal use for humans and/or animals internally, either orally or by other means and externally for, but not limited to, the following uses would dressings, poultices, salves, lotions, ointments and bio-active cosmetics and personal care products.

7. A METHOD OF ALTERING AND STANDARDIZING THE POTENCY AND BIOACTIVITY OF BIOACTIVE POLYSACCHARIDES comprising:
Selected minerals, either singly or in combination, are used together with a bioactive polysaccharide for additive and/or synergistic alteration of the bioactivity of the polysaccharide;
whereby measured amounts of a mineral, different minerals, different forms of minerals and/or blends of minerals used together with a quantity of a bioactive polysaccharide can cause the bio-active polysaccharide to produce substantially consistent and standardized effects; and

8. A MEANS UNDER CLAIM 7 OF ALTERING AND STANDARDIZING THE POTENCY AND BIOACTIVITY OF PURIFIED BIOACTIVE POLYSACCHARIDES comprising:
Selected minerals, either singly or in combination, are used together with a purified bioactive polysaccharide for additive and/or synergistic alteration of the bioactivity of the purified polysaccharide;
whereby measured amounts of a mineral, different minerals, different forms of minerals and/or blends of minerals used together with a quantity of a purified bio-active polysaccharide can cause the purified bioactive polysaccharide to produce substantially consistent and standardized effects; and

9. A MEANS UNDER CLAIM 7 OF ALTERING AND STANDARDIZING THE POTENCY AND BIOACTIVITY OF MAN MADE OR CULTURED BIOACTIVE POLYSACCHARIDES comprising:
Selected minerals, either singly or in combination, are used together with a man made or cultured bioactive polysaccharide for additive and/or synergistic alteration of the bioactivity of the man-made or cultured polysaccharide;
whereby measured amounts of a mineral, different minerals, different forms of minerals and/or blends of minerals used together with a quantity of a man made or cultured bioactive polysaccharide can cause the man-made or cultured bio-active polysaccharide to produce substantially consistent and standardized effects; and

10. A MEANS UNDER claim 7 OF ALTERING AND STANDARDIZING THE POTENCY AND BIOACTIVITY OF BIOACTIVE POLYSACCHARIDES CONTAINED IN HERBS AND PLANTS comprising:
Selected minerals, either singly or in combination, are used together with an herb or plant containing a bioactive polysaccharide for additive and/or synergistic alteration of the bio-activity of the polysaccharide contained in said herb or plant;
whereby measured amounts of a mineral,different minerals, different forms of minerals and/or blends of minerals used together with a quantity of an herb or plant containing a bio-active polysaccharide can cause the bioactivity of the polysaccharide to have substantially consistent and standardized effects; and

11. A MEANS UNDER CLAIM 7 OF ALTERING AND STANDARDIZING THE POTENCY AND BIOACTIVITY OF BIOACTIVE POLYSACCHARIDES CONTAINED IN HERBS AND PLANTS comprising:
Selected minerals, either singly or in combination, are used together with an herb or plant containing a bioactive polysaccharide for additive and/or synergistic alteration of the bioactivity of the polysaccharide contained in said herb or plant;
whereby measured amounts of a mineral or of different minerals or different forms of minerals such as, but not limited to, mineral elements, mineral salts, mineral colloids, mineral micro-clusters, atomic minerals, diatomic minerals, Ionic minerals, chelated minerals and other forms of minerals used singly or in combination, together with a given quantity of a bioactive polysaccharide can cause the bioactivity of the bioactive polysaccharide to be substantially consistent and standardized; and

12. A MEANS UNDER CLAIM 7 OF ALTERING AND STANDARDIZING THE POTENCY AND BIOACTIVITY OF BIOACTIVE POLYSACCHARIDES FOR HUMAN AND ANIMAL USE comprising:
Selected minerals, either singly or in combination, are used together with a bioactive polysaccharide for additive and/or synergistic alteration of the bioactivity of the polysaccharide;
whereby measured amounts of a mineral, different minerals, different forms of minerals and/or blends of minerals used together with a quantity of a bioactive polysaccharide can cause the bioactive polysaccharide to produce substantially consistent and standardized effects; and such a standardized bio-active polysaccharide would then be suitable for nutritive and/or medicinal use for humans and/or animals internally, either orally or by other means and externally for, but not limited to, the following uses: wound dressings, poultices, salves, lotions, ointments and bioactive cosmetics and personal care products.

13. A METHOD OF ALTERING AND CREATING VARIED AND/OR NEW BIOACTIVE PROPERTIES FOR BIOACTIVE POLYSACCHARIDES comprising:
Selected minerals, either singly or in combination, are used together with a bioactive polysaccharide for additive and/or synergistic alteration of the bioactivity of the polysaccharide;
whereby, a mineral, different minerals, different forms of minerals and/or blends of minerals used together with a bioactive polysaccharide can cause the bioactive polysaccharide to produce substantially varied and/or substantially new effects; and

14. A MEANS UNDER CLAIM 13 OF ALTERING AND CREATING VARIED AND/OR NEW BIOACTIVE PROPERTIES FOR PURIFIED BIOACTIVE POLYSACCHARIDES comprising:
Selected minerals, either singly or in combination, are used together with a purified bioactive polysaccharide for additive and/or synergistic alteration of the bioactivity of the purified polysaccharide;
whereby a mineral, different minerals,different forms of minerals and/or blends of minerals used together with a purified bioactive polysaccharide can cause the purified bioactive polysaccharide to produce substantially varied and/or substantially new effects; and

15. A MEANS UNDER CLAIM 13 OF ALTERING AND CREATING VARIED AND/OR NEW BIOACTIVE PROPERTIES FOR MAN-MADE OR CULTURED BIOACTIVE POLYSACCHARIDES comprising:
Selected minerals, either singly or in combination, are used together with a man made bioactive polysaccharide for additive and/or synergistic alteration of the bioactivity of the man made or cultured polysaccharide;
whereby a mineral, different minerals, different forms of minerals and/or blends of minerals used together with a man made or cultured bioactive polysaccharide can cause the man made or cultured bioactive polysaccharide to produce substantially varied and/or substantially new effects; and

16. A MEANS UNDER CLAIM 13 OF ALTERING AND CREATING VARIED AND/OR NEW BIOACTIVE PROPERTIES FOR THE BIOACTIVE POLYSACCHARIDES CONTAINED IN HERBS AND PLANTS comprising:
Selected minerals, either singly or in combination, are used together with an herb or plant containing a bioactive polysaccharide for additive and/or synergistic alteration of the bioactivity of the polysaccharide contained in said herb or plant;
whereby a mineral, different minerals, different forms of minerals and/or blends of minerals used together with an herb or plant containing a bioactive polysaccharide can cause the bioactive polysaccharide to produce substantially varied and/or substantially new effects; and

17. A MEANS UNDER CLAIM 13 OF ALTERING AND CREATING VARIED AND/OR NEW BIOACTIVE PROPERTIES FOR BIOACTIVE POLYSACCHARIDES comprising:
Selected minerals, either singly or in combination, are used together with a bioactive polysaccharide for additive and/or synergistic alteration of the bioactivity of the polysaccharide;
whereby a mineral, different minerals or different forms of minerals such as, but not limited to, mineral elements, mineral salts, mineral colloids, mineral micro-clusters, atomic minerals, diatomic minerals, ionic minerals, chelated minerals and other forms of minerals used singly or in combination, together with a bioactive polysaccharide can cause the bioactive polysaccharide to produce substantially varied and/or substantially new effects; and

18. A MEANS UNDER CLAIM 13 OF ALTERING AND STANDARDIZING THE POTENCY OF BIOACTIVE POLYSACCHARIDES FOR HUMAN AND ANIMAL USE comprising:
Selected minerals, either singly or in combination, are used together with a bioactive polysaccharide for additive and/or synergistic alteration of the bioactivity of the polysaccharide;
whereby a mineral, different minerals or different forms of minerals such as but not limited to mineral elements, mineral salts, mineral colloids, mineral micro-clusters, atomic minerals, diatomic minerals, Ionic minerals, chelated minerals and other forms of minerals used singly or in combination, together with a bioactive polysaccharide can cause the bioactive polysaccharide to produce substantially varied and/or substantially new effects and such an enhanced bioactive polysaccharide would then be suitable for nutritive and/or medicinal use for humans and/or animals internally, either orally or by other means and externally for, but not limited to, the following uses: wound dressings, poultices, salves, lotions, ointments and bioactive cosmetics and personal care products.
